(19) United States
(12) Patent Application Publication
(10) Pub. No.: US 2016/0058295 A1
(43) Pub. Date: Mar. 3, 2016

(54) PHOTOACOUSTIC WAVE MEASUREMENT APPARATUS AND PHOTOACOUSTIC WAVE MEASUREMENT METHOD

(71) Applicant: CANON KABUSHIKI KAISHA, Tokyo (JP)

(72) Inventors: Toru Imai, St. Louis, MO (US); Yasufumi Asao, Kyoto-shi (JP)

(21) Appl. No.: 14/838,214

(22) Filed: Aug. 27, 2015

(30) Foreign Application Priority Data
Sep. 2, 2014 (JP) 2014-178561

(51) Int. Cl. A61B 5/00

(52) U.S. Cl. CPC A61B 5/0095; A61B 5/742

(57) ABSTRACT

A measurement region of an object on which irradiation of light is performed and detection of photoacoustic waves is controlled is set based on positional information of a specific region within a captured surface image of the object.

### BACKGROUND OF THE INVENTION

1. Field of the Invention

The present invention relates to a photoacoustic wave measurement apparatus and a photoacoustic wave measurement method.

2. Description of the Related Art

An optical imaging apparatus causes light, irradiated from a light source such as laser, to be propagated through a subject and detects a signal based on the propagated light to thereby acquire information on the inside of the subject. This technique has been actively studied in medical fields as an in vivo examination technique. Photoacoustic imaging is known as one of such optical imaging technologies.

Photoacoustic imaging is a technology which irradiates pulsed light generated from a light source to a subject, detects acoustic waves (hereinafter, also referred to as “photoacoustic waves”) generated from a biological tissue having absorbed energy of light diffused within the subject, and visualizes information relating to an optical characteristic value of inside of the subject. Thus, an optical characteristic value distribution of inside of the subject, particularly, a light energy absorption density distribution may be acquired.

Using light having a wavelength which may be absorbed by hemoglobin as the pulsed light, for example, allows non-invasive imaging of an angiogram of inside of a living body. Using light having a wavelength different from that absorbed by hemoglobin allows imaging of other parameters such as collagen and elastin under the skin. The use of a contrast medium responsive to a specific wavelength also allows highlighting of an angiogram and imaging of a lymphatic vessel.

A representative technology of three-dimensional visualization technologies applying photoacoustic imaging detects photoacoustic waves generated from a light absorber by using an ultrasonic transducer placed on a two-dimensional plane and performs image reconstruction computing thereon to generate three-dimensional data relating to optical characteristic values. Such a three-dimensional visualization technology is called a photoacoustic tomography (PAT).

Furthermore, in recent years, a photoacoustic microscope attracts attention as an apparatus which allows visualization with a high spatial resolution by applying the photoacoustic imaging. A photoacoustic microscope uses an optical lens or an acoustic lens to focus light or sound so that a high-resolution image may be acquired.

However, it is known that the depth for visualization and the spatial resolution are trade-offs in an apparatus using PAT and a photoacoustic apparatus such as a photoacoustic microscope. In other words, PAT has a feature that information acquired by PAT has a spatial resolution which decreases as the depth of tissue of a living body increases. This is because light is easily diffused within a living body and photoacoustic waves at a high frequency generated from the living body attenuate largely as the depth of tissue of a living body increases. Because of this characteristic, a photoacoustic microscope with a high spatial resolution, for example, is mainly applicable for visualizing a light absorber within the skin present in a relatively shallower part of a living body. For example, in a case where a photoacoustic microscope may be used to visualize blood hemoglobin, a blood vessel present in a dermal layer of the skin may be visualized. A non-patent literature “In vivo dark-field reflection-mode photoacoustic microscopy,” by Maslov et al., OPTICS LETTERS Vol. 30, No. 6, March 2005, discloses a photoacoustic microscope capable of capturing at a high resolution an image of a blood vessel present in a region close to the skin by using an acoustic lens.

Generally, a mole or a body hair present on the skin or in an epidermal layer exhibit a high light absorbing quantity. A “mole” is a cluster of pigmented cells called melanocytes. Moles, freckles, skin tags, seborrheic keratosis, and lentigines are formed when pigmented cells grow in clusters on the skin. Thus, photoacoustic waves generated based on light absorbed by a mole or a body hair have a higher intensity than that of photoacoustic waves acquired from other regions of the skin. Therefore, using the photoacoustic microscope disclosed in the above-mentioned non-patent literature may provide wrong information from photoacoustic waves based on a mole or a body hair, which may possibly prevent correct evaluation of biological information of a dermal layer present under a cluster of pigmented cells or body hair.

### SUMMARY OF THE INVENTION

Accordingly, an aspect of the present invention provides a photoacoustic apparatus capable of properly visualizing information relating to optical characteristic values of a tissue to be visualized even in a case where an object which highly absorbs light, such as a mole or body hair, exists on the skin or in an epidermal layer.

A photoacoustic wave measurement apparatus according to one aspect of the present invention includes an image pickup unit configured to capture a surface image of an object, a light source configured to generate light, a photoacoustic wave detection unit configured to detect photoacoustic waves propagated from an object irradiated with the light, a specific region detection unit configured to detect a specific region within the captured surface image of the object, and a control unit which sets a measurement region of the object to perform irradiation of the light and controls detection of the photoacoustic waves based on positional information of the specific region detected by the specific region detection unit.

A photoacoustic wave measurement apparatus according to another aspect of the present invention includes an image pickup unit, a light source configured to generate light, a photoacoustic wave detection unit configured to detect photoacoustic waves propagated from an object irradiated with the light, a specific region detection unit configured to detect a specific region within a captured image obtained by the image pickup unit, and an image processing unit that generates image data from the photoacoustic waves detected by the photoacoustic wave detection unit based on positional information of the specific region detected by the specific region detection unit.

A photoacoustic wave measurement method according to still another aspect of the present invention includes obtaining a captured image of an object, detecting a specific region within the captured image, setting a measurement region of the object based on a position of the specific region within the captured image, and irradiating light to the measurement region and detecting photoacoustic waves propagated from the measurement region of the object in response to the irradiation of the light.

A photoacoustic wave measurement method according to still another aspect of the present invention includes obtaining a captured image of an object, detecting a specific region within the captured image, irradiating light to the object and detecting photoacoustic waves propagated from the object in response to the irradiation of the light, and generating image data from the detected photoacoustic waves based on a position of the specific region within the captured image.

Further features of the present invention will become apparent from the following description of exemplary embodiments with reference to the attached drawings.

### BRIEF DESCRIPTION OF THE DRAWINGS

FIG. 1 illustrates a configuration of a photoacoustic measurement apparatus according to an embodiment of the present invention.

FIG. 2 is a flowchart for measurement to be performed by a photoacoustic measurement apparatus according to an embodiment of the present invention.

FIG. 3 is a flowchart for measurement to be performed by a photoacoustic measurement apparatus according to an embodiment of the present invention.

FIG. 4 is a timing chart for measurement to be performed by a photoacoustic measurement apparatus according to an embodiment of the present invention.

FIG. 5 illustrates wavelength dependencies of reflectances of a subject surface tissue excluding a non-measuring tissue and the non-measuring tissue.

FIG. 6 illustrates wavelength dependencies at signal levels on captured images of a subject surface tissue excluding a non-measuring tissue and the non-measuring tissue.

FIGS. 7A and 7B are diagrams for explaining a method for storing correspondence data between signal levels on a captured image and wavelengths of illumination light and data on correlation coefficients according to an embodiment of the present invention.

FIGS. 8A and 8B illustrate a method for setting a measurement region according to an embodiment of the present invention.

FIG. 9 illustrates a configuration of a photoacoustic measurement apparatus according to an embodiment of the present invention.

FIG. 10 is a flowchart for measurement to be performed by a photoacoustic measurement apparatus according to an embodiment of the present invention.

FIGS. 11A and 11B are diagrams for explaining a method for excluding measurement data according to an embodiment of the present invention.

FIG. 12 illustrates a configuration of a photoacoustic measurement apparatus according to an embodiment of the present invention.

FIG. 13 is a flowchart for measurement to be performed by a photoacoustic measurement apparatus according to an embodiment of the present invention.

FIGS. 14A and 14B illustrate a method for excluding measurement data according to an embodiment of the present invention.

### DESCRIPTION OF THE EMBODIMENTS

### First Embodiment

A photoacoustic wave measurement apparatus according to a first embodiment is an ultrasonic-focus photoacoustic microscope, for example. According to this embodiment, an ultrasonic-focus photoacoustic microscope refers to a photoacoustic microscope configured such that a focal point region of pulsed light may be wider than a focal point region of ultrasound. The present invention is applicable not only to an ultrasonic-focus photoacoustic microscope but also to a light-focus photoacoustic microscope configured such that a focal point region of pulsed light may be smaller than a focal point region of ultrasound.

An example of material to be visualized by photoacoustic measurement is tissue or a phantom having light absorption coefficient and scattering coefficient equivalent to tissue. An example of the specific region reference material is a pre-identified non-measuring tissue of photoacoustic measurement according to this embodiment. According to this embodiment, in a region where photoacoustic measurement is to be executed, pulsed light 104 is irradiated to the region, and photoacoustic waves are detected. On the other hand, in a region where photoacoustic measurement is not executed, at least one of irradiation of pulsed light 104 and photoacoustic wave detection is not performed.

### Overall Configuration of Photoacoustic Wave Measurement Apparatus

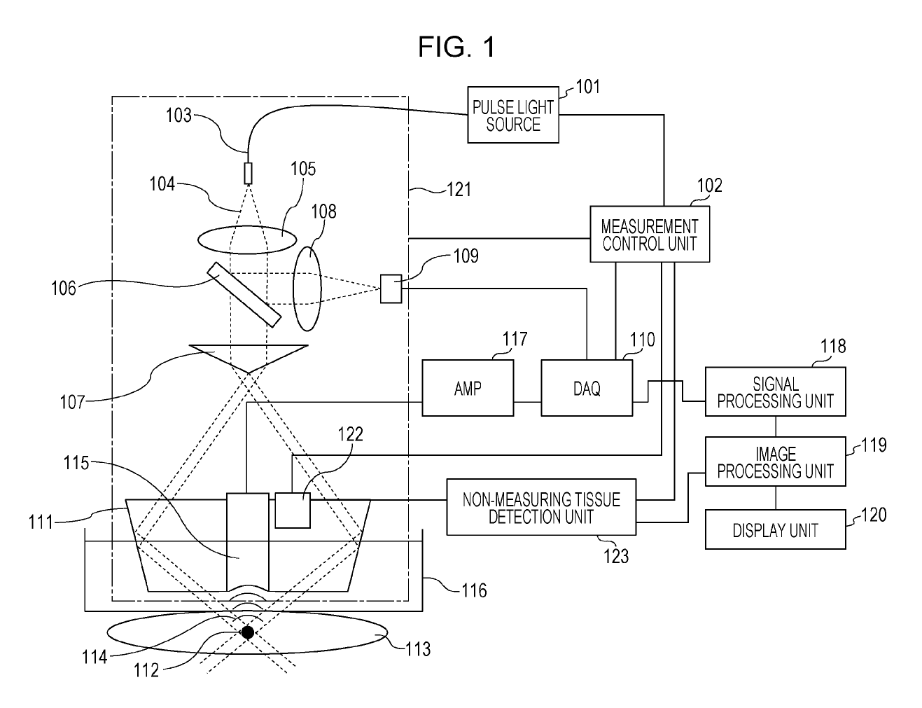

An overall configuration of a photoacoustic microscope, an example of photoacoustic wave measurement apparatus, according to this embodiment will be described. FIG. 1 illustrates an overall configuration of an ultrasonic-focus photoacoustic microscope.

A pulsed light source 101 emits pulsed light under control of a measurement control unit 102. The pulsed light passes through an optical fiber 103 and is guided to an optical system for irradiating excited light to a living body. According to this embodiment, the optical system includes a lens 105, a beam splitter 106, a conical lens 107, and a lens 108. The pulsed light 104 emitted from the optical fiber 103 is collimated by the lens 105, and a part of the light passes through the beam splitter 106 and another part of the light is reflected by the beam splitter 106. The pulsed light having passed through the beam splitter 106 spread in a ring shape by the conical lens 107 and is launched into a mirror 111. On the other hand, the pulsed light reflected by the beam splitter 106 is collected by a lens 108 and is detected by a photodetector 109. A digital signal acquired by converting the detected signal by a data acquisition unit (DAQ unit) 110 is stored in an internal memory of the DAQ unit. The pulsed light detected by the photodetector 109 may be used for correcting an error due to a variation in light quantity of the photoacoustic signal and may be usable as a trigger signal for determining measurement timing of photoacoustic waves.

The pulsed light 104 spreads into a ring shape by the conical lens 107, and is then collected by being reflected by the mirror 111. The mirror 111 mainly contains a member made of transparent material such as glass and is configured to reflect the pulsed light 104 at the boundary between the mirror 111 and the outside of the mirror 111 (such as air or water, which will be described below). A metal film may be evaporated on the outer surface of the member forming the mirror 111 for a higher light reflectance. The mirror 11 is set such that the position of the focal point of light collected by the mirror 111 may be located in an internal part of the subject 113 for measurement of photoacoustic waves. According to this embodiment, the lens 105, conical lens 107, and mirror 111 function as an optical unit configured to guide the pulsed light 104 to the subject 113. The pulsed light diffused by an internal part of the subject 113 is absorbed by a light absorber 112 (e.g., blood) located within the subject 113. Some types of light absorber 112 may have a specific or predetermined light absorption coefficient. When the light absorber 112 absorbs light, photoacoustic waves 114 are generated due to the well-known photoacoustic effect. The photoacoustic waves 114 are detected by a transducer 115 functioning as a photoacoustic wave detection unit placed near the center of the mirror 111, and a change in sound pressure intensity is converted to an electric signal. The transducer 115 includes an ultrasonic transducer (of one or more transducer elements) sensitive to an ultrasonic frequency band. The transducer 115 may have an acoustic lens. According to the present embodiment, the transducer 115 having an acoustic lens is capable of detecting sound waves generated from its focal position with high sensitivity. The focal point of the acoustic lens may be set to the focal position of the pulsed light collected by the mirror 111 so that sound waves generated from the focal position of the pulsed light may be detected with high sensitivity. Water stored in a tank 116 is present between the transducer 115 and the subject 113 for acoustic impedance matching with the subject. The material for acoustic impedance matching to be stored in the tank 116 is not limited to water but may contain other substances. A gel acoustic impedance matching material may be coated between a bottom of the tank 116 and the subject 113. The photoacoustic waves 114 are detected by the transducer 115 and are converted to an electric signal. The electric signal acquired by the transducer 115 is transmitted to a signal amplifier 117, and its signal intensity is amplified. After that, the electric signal is converted to a digital signal by the DAQ unit 110 and is stored in an internal memory of the DAQ unit 110. The data stored in the DAQ unit 110 undergo signal processing performed by the signal processing unit 118. After that, the data undergo image processing performed by the image processing unit 119 and are displayed as image data by the display unit 120. The signal processing unit 118 and image processing unit 119 may be configured as an integral processing unit.

According to the present embodiment, members enclosed within a box 121 are mounted on a movable stage (not illustrated) capable of scanning two-dimensionally. Two-dimensional relative movement of the movable stage about the subject 113 may move positions of the focal point of the pulsed light 104 focused on the subject 113 and the focal point of the transducer 115. By detecting photoacoustic waves at two-dimensionally scanned measurement positions, two-dimensional photoacoustic signal data of the subject may be acquired.

An image pickup unit (camera) 122 is further provided adjacent to the transducer 115 for capturing a surface image of the subject 113. The mirror 111 including a transparent member for a wavelength band of light to which the image pickup unit 122 is sensitive may acquire an image of a surface of the subject 113. The illumination for capturing an image of the subject 113 may be light from an external environment or light generated from the pulsed light source 101. Alternatively, an illumination unit may be provided separately from the pulsed light source 101, and the illumination unit may be used to illuminate the subject 113. In a case where the pulsed light source 101 is used as an illumination unit for image capturing, light having an identical wavelength characteristic to that of the light generated when a photoacoustic measurement is performed may be used as the illumination light. Apparently, light having a wavelength characteristic different from that of light generated when a photoacoustic measurement is performed may be used as illumination light. When image data acquired by the image pickup unit 122 is transmitted to a specific region detection unit 123, the specific region detection unit 123 detects data on a non-measuring tissue of a photoacoustic measurement among image data. Based on the detected data, the measurement control unit 102 functioning as a control unit sets a region where a photoacoustic measurement is to be executed.

Referring to FIG. 1, the image pickup unit 122 is embedded in the mirror 111. However, the image pickup unit 122 may be retained separately and be moved to above the subject 113 for capturing a surface image of the subject 113.

The measurement control unit 102 may perform light emission control of the pulsed light source 101, control over the movable stage (not illustrated), control regarding data sampling of the DAQ unit 110, control over image capturing performed by the image pickup unit 122 and illumination therefor and so on.

In the aforementioned configuration, the focal point region of the pulsed light 104 contains a focal point of ultrasonic waves of an acoustic lens included in the transducer 115, for example. However, the relationship may be reversed, as in the configuration of a light-focus photoacoustic microscope. In other words, a focal point region of focus of the pulsed light 104 through an objective lens may contain a region of a focal point of ultrasonic waves of the acoustic lens in the transducer 115. When a light-focus photoacoustic microscope is used, the resolution of the photoacoustic microscope depends on the size of the focal point of light. Thus, a photoacoustic image at a higher resolution may be acquired.

### Operational Timing

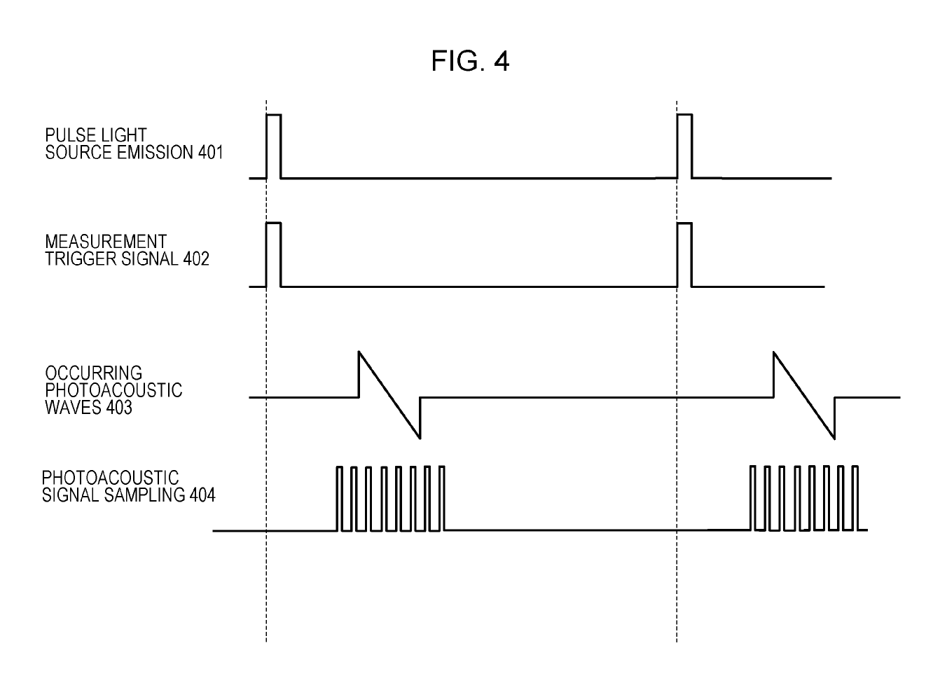

Next, with reference to the timing chart in FIG. 4, timing of operations in a measurement of a photoacoustic signal will be described. For easy description, FIG. 4 illustrates signal shapes and time points simply. FIG. 4 illustrates light emission timing 401 of the pulsed light source 101. According to the present embodiment, light emission timing of the pulsed light source is controlled by the measurement control unit 102. However, for example, the light emission timing may be controlled based on trigger signals output at equal moving distance intervals or equal time periods from an encoder on a movable stage, not illustrated. FIG. 4 further illustrates a measurement trigger signal 402 generated from pulsed light detected by the photodetector 109 for starting a measurement of a photoacoustic signal. The trigger signal may refer to a signal which controls the light emission timing of the pulsed light source or may refer to the pulsed light 104 reflected by the beam splitter 106, as described above. FIG. 4 further illustrates photoacoustic waves 403 which are excited by pulsed light generated from the pulsed light source 101 at time points exhibited in the signal 401 and reaches the transducer 115. The photoacoustic waves detected by the transducer 115 delay by a time period from the source to the transducer 115 with respect to the light emission timing of the pulsed light source 101. A signal 404 exhibits sampling timing for measuring a photoacoustic wave reaching the transducer 115. Sampling starts from a time delayed from the measurement trigger signal is performed over a period necessary for imaging photoacoustic waves. According to the present embodiment, a delay time is provided from a light emission time point of the pulsed light source 101 to the start of the sampling of photoacoustic waves. However, if the DAQ unit 110 has adequate memory, the sampling may start in synchronism with light emission of the pulsed light source 101 without delay. The measurement sampling frequency may be set to at least a double frequency of the main frequency of generated photoacoustic waves as high as possible.

### Data Acquisition Process

Next, a method will be described more specifically which uses the ultrasonic-focus photoacoustic microscope as described above to acquire two-dimensional plane data of a photoacoustic signal generated from inside of the subject 113 and display a corresponding image.

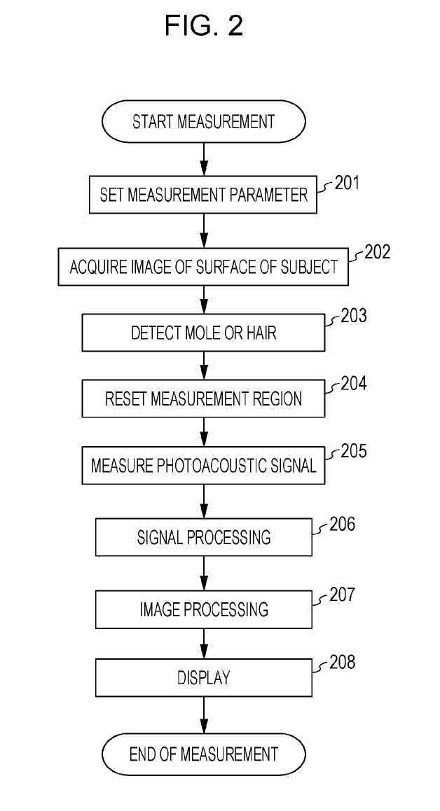

A measurement procedure will be described with reference to the flowchart for a measurement in FIG. 2. Before a measurement, a subject 113 is placed from which a photoacoustic image is measured by using the ultrasonic-focus photoacoustic microscope. After the subject is placed, a measurement flow starts.

In step 201, a measurement parameter such as a measurement region for a photoacoustic measurement is set in the measurement control unit 102. Other concrete examples of the measurement parameter may include a measurement pitch of a photoacoustic measurement, a storage/sampling frequency of a photoacoustic signal at one point, a storage time, a scanning rate of a movable stage, acceleration, a light emission frequency of the pulsed light source 101, a light quantity, and a wavelength.

In step 202, an image of a surface of the subject 113 is acquired. The movable stage is moved based on information on the measurement region set in step 201, and the subject 113 is captured by the image pickup unit 122 such that the measurement region for acquiring a photoacoustic image may be included to acquire image data. The moving amount of the movable stage is calculated in consideration of a relative distance between the positions where the image pickup unit 122 and the transducer 115 are placed. Data on a surface image of the subject 113 captured by the image pickup unit 122 are transmitted to the specific region detection unit 123.

In step 203, a mole or a body hair present on a surface of the subject 113 are detected. The specific region detection unit 123 locates a position on a captured image of a tissue of a mole or a body hair present on a subject surface. In this case, the tissue has a significant light absorbing quantity from blood hemoglobin to be visualized by a photoacoustic measurement. A specific method for detecting a mole or a body hair from a captured image will be described below.

In step 204, the photoacoustic measurement region is reset. The measurement control unit 102 adjusts the alignment of the positions of the image of the subject surface and the photoacoustic measurement region set in step 201 and then resets a new photoacoustic measurement region excluding a position of at least one of a mole and a body hair detected in step 203. In step 201, the setting of a measurement region may be omitted. A photoacoustic wave measurement region may be set in step 204.

The method for adjustment of the alignment of the positions may be implemented by grasping coordinates of the image pickup unit 122 and transducer 115 and an imaging region of the image pickup unit with reference to the coordinates managed within the movable stage. Also in a case where no camera is mounted on the movable stage, the alignment of a captured image and a photoacoustic measurement region may be adjusted with reference to a subject or a marking around the subject. Details of the method for re setting a photoacoustic measurement region will be described below.

In step 205, based on the measurement region reset in step 204, a photoacoustic signal is acquired for each measurement position within the measurement region. Operations for a photoacoustic measurement are performed as described above.

Acquisition of a photoacoustic signal at each measurement position will be described. Through the procedure described with reference to FIG. 2, the photoacoustic wave 114 generated from the subject is converted to an electric signal by the transducer 115. The acquired electric signal is amplified by the signal amplifier 117. Then, the amplified electric signal is converted to a digital signal by the DAQ unit 110 and is stored in an internal memory of the DAQ unit 110. Data stored in the DAQ unit 110 are transmitted to the signal processing unit 118 under control of the measurement control unit 102. When measurements are performed completely at all measurement positions by moving the movable stage throughout the measurement positions, the acquisition of the photoacoustic signals completes.

In step 206, signal processing is performed by the signal processing unit 118 on the electric signals based on the photoacoustic signals acquired at the measurement positions. Specific processes of the signal processing may include deconvolution in consideration of a pulse width of the pulsed light source 101 and envelop line detection. In a case where a distinctive frequency of noise added to a signal is known in advance and can be separated from a main frequency of a photoacoustic signal, a specific frequency component due to noise can also be removed. A component based on a photoacoustic wave reaching the transducer 115 with a delay from a wave directly transmitted from a sound source of a photoacoustic wave due to reflection by a surface of the subject 113 or a bottom of the tank 116 may be removed from the signal. In a case where a component based on a photoacoustic wave generated on a surface of the subject 113 is significant, the component may be deleted by performing this flow.

In step 207, image processing is performed. The image processing unit 119 generates voxel data based on a position on a scanning plane of the movable stage and a signal intensity distribution in a depth direction of a subject of a photoacoustic signal having undergone the signal processing in step 206 and generates an image data for visualization. In this case, a known artifact if any may be removed from the voxel data. In a case where an oxygen saturation of a light absorber within a subject is calculated, for example, voxel data storing oxygen saturation values may be generated from voxel data on intensities of photoacoustic signals acquired at a plurality of wavelengths of pulsed light. In a case where the measurement is performed by setting a wavelength of pulsed light such that blood hemoglobin within a subject may be a main light absorber, an image of a blood vessel is binarized and extracted from the acquired voxel data, for example.

In step 208, voxel data generated from the intensity distribution of photoacoustic signals in step 207 are displayed by a display method desired by a user. For example, the voxel data may be displayed by a method which displays a vertical cross section on three-dimensional axes or a method which displays voxel data as a two-dimensional distribution of the highest value, minimum value or average value of the voxel data in axis directions. Alternatively, a user may set an ROI (region of interest) within voxel data, and a user interface program may be configured to display statistical information regarding a shape of light absorber within the region and oxygen saturation information. In this case, in step 204, the value calculated based on a photoacoustic measurement does not exist with respect to the region from which a mole or a body hair is detected and thus excluded from the measurement region. Therefore, calculations are performed with reference to a region excluding the region from which a mole or body hair is detected. Dummy data on a region having a mole or a body hair excluded from the measurement region in step 204 may be displayed on the display unit 120.

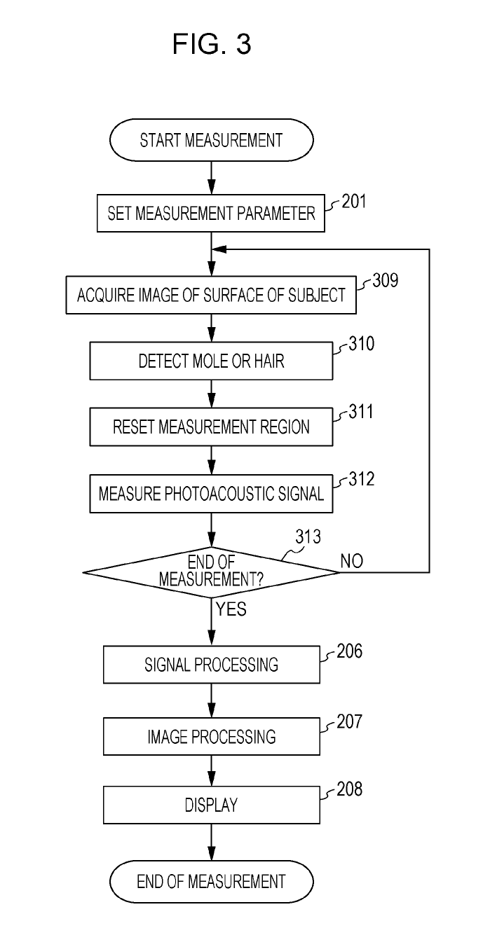

In the measurement flow as described above, after whether a photoacoustic measurement is to be performed over all of the measurement region set in step 201 or not is determined, a measurement region is reset, and a photoacoustic measurement is performed thereon. However, during the photoacoustic measurement step 205, scanning may be performed with a movable stage capable of moving relatively to the subject 113, and the determination on whether a measurement is to be performed or not (and resetting of a measurement region) may be performed on positions one by one. The determination may be repeated until the scanning on the measurement region set in step 201 ends. FIG. 3 illustrates this flow. In step 309, an image of a region including a position on which the next photoacoustic-wave measurement is to be performed. In step 310, a mole or a body hair, for example, is detected on the region from which an image is acquired. In step 311, whether a photoacoustic wave measurement is to be executed on the next measurement position or not is determined based on the detection result, and the measurement region is reset. In step 312, based on details of the resetting of the measurement region, a photoacoustic-wave measurement is executed. In step 313, whether all measurements have completed or not is determined. If not, the same steps are repeated. The subsequent steps are performed as in the flow in FIG. 2.

### Specifying Non-Measuring Region

Next, a specific method will be described which detects a non-measuring tissue of the photoacoustic measurements present on a subject surface and excludes it from a measurement region of a photoacoustic measurement. For example, this method is applicable to a case where a mole or a body hair, for example, having a high light absorbing quantity on the skin surface is excluded from a measurement region in order to image a blood vessel under the skin by performing a photoacoustic measurement.

According to the present embodiment, a camera is used as an image pickup unit for acquiring a surface image of the subject 113. The image pickup unit may be an apparatus including a two-dimensional array type sensor in which pixels are arranged two-dimensionally.

The surface image may be acquired by using one kind of monochromatic light as illumination light or may be acquired for each of a plurality of kinds of monochromatic light switched to irradiate the subject 113.

First, a unit for using a monochromatic wavelength as the illumination light will be described.

Assume a case in which a non-measuring tissue is identified in advance to the measurement. Here, such a non-measuring tissue has a significantly higher light absorbing quantity than that of a tissue to be visualized by performing a photoacoustic measurement. For example, it may correspond to a mole or a body hair on the skin when an object of the visualization by photoacoustic measurement is blood hemoglobin. A difference in light absorbing quantity between a tissue to be visualized by performing a photoacoustic measurement and another tissue may be acquired by, for example, acquiring photoacoustic signals of tissue samples thereof and comparing the light absorbing quantities at a specific wavelength based on their signal intensities. Alternatively, a non-measuring tissue may be identified from information on known light absorption coefficients of the tissues.

In a case where one kind of monochromatic light is used as the illumination light, a wavelength of the illumination light may be selected which provides sufficient contrast on a captured image between a non-measuring tissue of a photoacoustic measurement and a subject surface tissue which is an object.

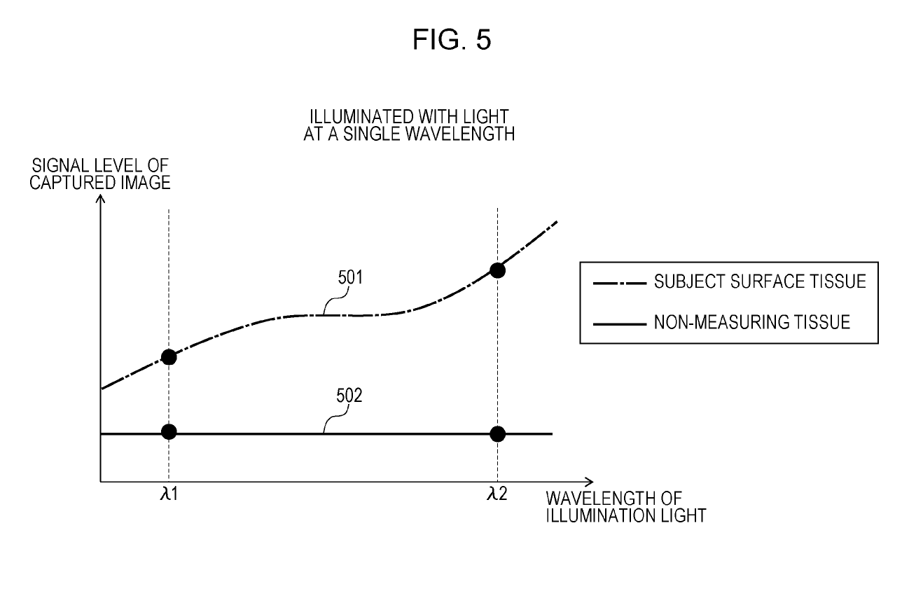

FIG. 5 illustrates examples of wavelength characteristics of illumination light having the intensities on the captured image of the surfaces of the two tissues. FIG. 5 illustrates a wavelength dependency 501 of the intensity on a captured image of the subject surface tissue which is an object and a wavelength dependency 502 of the intensity on the captured image of a non-measuring tissue such as a mole or a body hair on the skin. In the example illustrated in FIG. 5, a wavelength λ2 providing a large difference between intensities on a captured image of the two tissues of wavelengths λ1 and λ2 is used as the illumination light. This is because selecting light having the wavelength λ2 as the illumination light provides an image having a higher contrast between the tissues compared with a case where light with the wavelength λ1 is used.

In order to acquire wavelength dependencies of intensities on a captured image of tissues, images of the tissues may be captured previously, or the wavelength dependencies may be calculated in consideration of wavelength dependencies of reflectances of the tissues and a wavelength dependency of an optical element of a camera to be used. In a case where the wavelength dependencies of images are acquired by performing a calculation or an experiment, an incident angle dependency of illumination light is also considered as required. Alternatively, a diffusing plate may be used for a lower incident angle dependency to diffuse and irradiate illumination light to a subject.

Also in a case where a non-measuring tissue is not identified previously, the tissue may be detected from an image captured by using illumination light having a monochromatic wavelength. The method will be described below.

An example will be described in which a non-measuring tissue of a photoacoustic measurement has a relatively higher light absorbing quantity, on a surface of the subject 113, at a wavelength of pulsed light to be irradiated to the subject 113 for a photoacoustic measurement. A tissue having a relatively higher light absorption at a certain wavelength than that of a surrounding tissue has a lower reflectance at the wavelength than that of the surrounding tissue. Therefore, its luminance on a captured image is relatively low, that is, its signal level is relatively low. For example, in a case where the wavelength to be used for a photoacoustic measurement is in a visible light region and light having the wavelength is used as illumination for capturing an image, a mole or a body hair generally has a higher light absorbing quantity than that of surrounding skin. Thus, signal levels of the pigmented tissues, e.g., where a cluster of pigmented cells called melanocytes are captured in an image, are relatively lower than the surrounding skin. Therefore, a captured image can be acquired which has a signal level dependent on the magnitude of the light absorbing quantity at a wavelength used for a photoacoustic measurement, which allows detection of a non-measuring tissue.

Next, a case will be described in which a non-measuring tissue of photoacoustic measurements has a significantly higher light absorbing quantity than that of an object of visualization by the photoacoustic measurements. In this case, a material to be visualized in the photoacoustic measurements includes a tissue or a prepared phantom having an equal light absorption coefficient and scattering coefficient as that of the tissue. To that end, light having a wavelength used for the photoacoustic measurements may be used as illumination light to acquire in advance the signal level on the captured image. The prepared signal level corresponding to the tissue to be visualized among captured images of a subject surface in the measurement region are defined as a threshold to identify a region having an intensity equal to or lower than the threshold as a non-measuring region. Thus, at a wavelength usable for performing the photoacoustic measurements, a region having a tissue having a higher light absorbing quantity than the light absorbing quantity of the tissue to be visualized can be identified as a non-measuring region of the photoacoustic measurements. The opposite may also be true. That is, a region of tissue having a lower light absorbing quantity than the light absorbing quantity of the tissue to be visualized can be identified as a non-measuring region of the photoacoustic measurements.

Next, a method will be described which uses illumination light having a plurality of monochromatic wavelengths to identify the position of a non-measuring tissue of the photoacoustic measurements. In a case where illumination light having a plurality of monochromatic wavelengths is used, the kind of non-measuring tissue of photoacoustic measurements may be identified in advance. For example, when a region having a mole is a non-measuring tissue of photoacoustic measurements, the wavelength dependency of the intensity of the reflected light from the mole may be known in advance. For example, an image of a subject surface tissue including a mole is captured for each wavelength of illumination light having a plurality of monochromatic wavelengths, and the signal level of the region having the mole on the captured image is acquired for each wavelength of the illumination light. Thus, the dependency of the mole on the wavelengths of the illumination light may be acquired a priori. When the dependency of the mole on the wavelengths of the illumination light is unknown, the wavelength dependency of the reflectance of the mole and the wavelength dependency of the signal levels of the captured image may be possible to be obtained by calculation or experimentation.

After the data on the wavelength dependency of the mole with respect to the illumination light on the captured image are prepared, images of a subject surface may be captured by using illumination light having a plurality of monochromatic wavelengths so that a signal level of each of pixels of the captured image for each of the wavelengths may be acquired. After that, a correlation coefficient for each pixel is calculated between the acquired signal level on the captured image and the prepared data on the wavelength dependency of the mole on the captured image with respect to the illumination light. For example, as a result of the calculation of the correlation coefficient by using the prepared data on the wavelength dependency of the mode on the captured image, it may be considered that there is a high possibility that a pixel having a high correlation coefficient is a result of the capturing an image of the mole. This may give high contrast between a non-measuring mole of photoacoustic measurements and the other subject surface tissue within a captured image for identification. When a subject is illuminated with light at different wavelengths, the light quantity of the illumination light may be monitored to correct the light quantity and to correct a signal level on a captured image.

Having described above the example in which a correlation coefficient is calculated for each pixel of a captured image, the correlation coefficient may be calculated based on a value acquired by adding or averaging signal levels of a plurality of pixels.

The method has been described above for identifying a non-measuring tissue of photoacoustic measurements by using a mole as an example. On the other hand, for a tissue on which photoacoustic measurements are to be performed, wavelength dependency data on a captured image may be prepared to identify a region on which photoacoustic measurements are to be performed and a non-measuring region of photoacoustic measurements.

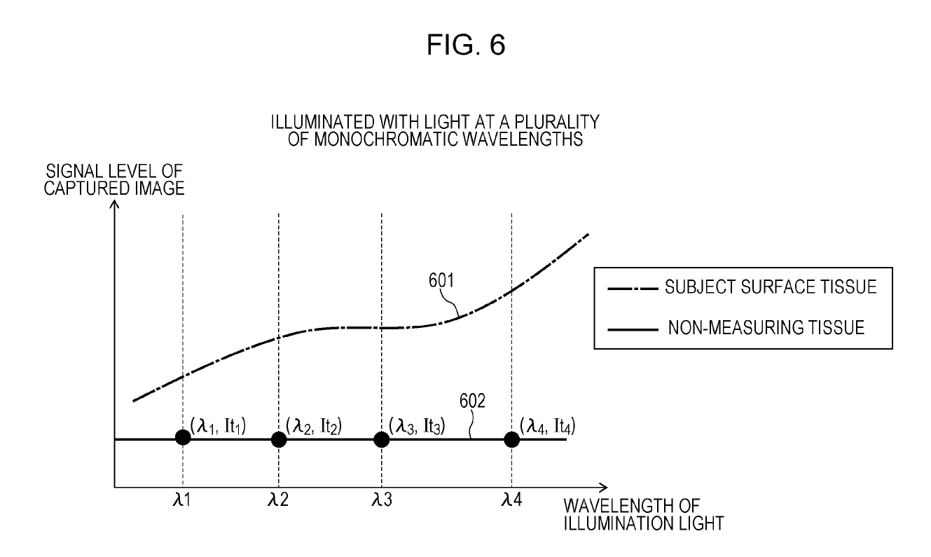

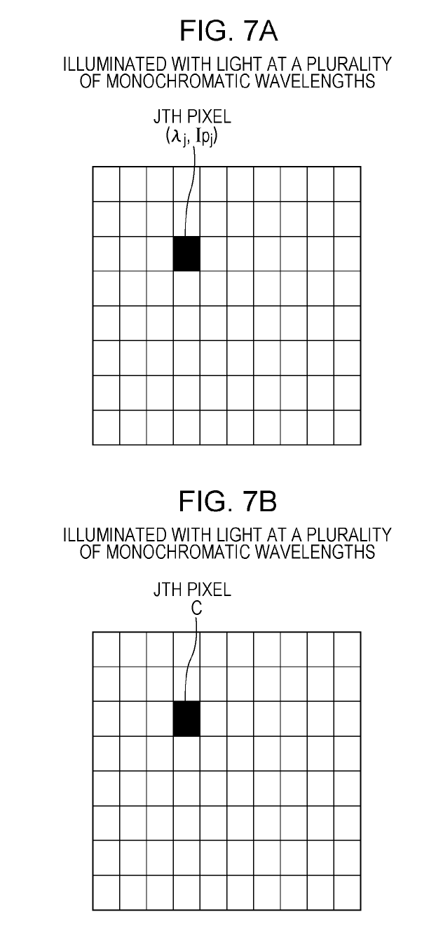

Next, supplementary explanation will be given on the method with reference to drawings. FIG. 6 illustrates example wavelength dependencies 601 and 602 of intensities on a captured image of a subject surface tissue (or a tissue excluding a non-measuring tissue) and the non-measuring tissue, respectively. As described above, for example, combination data (λᵢ,Itᵢ) of wavelengths and image intensities in a case where a non-measuring tissue is illuminated with light at four wavelengths as illustrated in FIG. 6 are prepared in advance. The subscript i indicates a wavelength used as illumination light. After that, image pickup unit 122 uses illumination light at a plurality of monochromatic wavelengths to capture an image of a measurement region of a subject. FIGS. 7A and 7B illustrates pixels of a captured image in a grid-like manner. As illustrated in FIG. 7A, the combination data (λᵢ,Ipᵢ) of wavelengths of illumination light and signal levels on a captured image are stored in a memory in association with pixels. FIGS. 7A and 7B illustrate data stored in association with the Jth pixel. A correlation coefficient C of prepared data on a signal level of a mole on a captured image and the data on an image intensity stored for each pixel illustrated in FIG. 7A is calculated and is stored in the memory for each pixel. In other words, a correlation coefficient C is applied to the Jth pixel as illustrated in FIG. 7B. The correlation coefficient is calculated by using the following Expression (1). In other words, a plurality of kinds of light having wavelength characteristics different from each other are used as illumination light, and images of an object are captured in a state of irradiating the kinds of light to the object. From the thus acquired plurality of images, the following correlation coefficient is calculated.

$$C = \frac{{\sum }_{k = 1}^{n} ( {Ip}_{k} - \overline{I} {p}_{k} ) ( {It}_{k} - \overline{I} {t}_{k} )}{\sqrt{{\sum }_{k = 1}^{n} {( {Ip}_{k} - \overline{I} {p}_{k} )}^{2}} \sqrt{{\sum }_{k = 1}^{n} {( {It}_{k} - \overline{I} {t}_{k} )}^{2}}} \qquad ( 1 )$$

In this case, n is the number of used wavelengths and is an integer equal to or higher than 2. Thus, a pixel having a high correlation coefficient with a wavelength dependency of a mole is highly possibly a pixel corresponding to the mole on the captured image data. Therefore, the mole may be detected from the captured image.

This method allows acquisition of image data displaying a non-measuring biological tissue of photoacoustic measurements and a biological tissue surface which is an object at a sufficiently high contrast ratio. Thus, the position of a non-measuring biological tissue of photoacoustic measurements may be identified from an image captured by an image pickup unit. For some characteristics of biological tissues, the aforementioned methods may be used in combination to secure a sufficiently high contrast ratio.

The method has been described which uses light having a single monochromatic wavelength or a plurality of monochromatic wavelengths as illumination light if the aforementioned methods use such light. However, a subject may be irradiated with non-monochromatic light as illumination light such as light having a broad wavelength band. In this case, an optical filter allowing only light having a monochromatic wavelength to pass through may be used.

Therefore, the illumination light is not limited to light having a monochromatic wavelength but may be light having a broad wavelength characteristic such as white light. For example, the illumination light may be light in a room where the ultrasonic-focus photoacoustic microscope according to the present embodiment is placed. Alternatively, if a camera includes a white light illumination, the provided white light illumination may be used. A sensor capable of outputting in color may be used as a sensor of the camera. In this illumination method, a non-measuring tissue of photoacoustic measurements is identified in advance.

Next, a method will be described which extracts pixels capturing a non-measuring tissue of photoacoustic measurements from a captured image of a subject surface acquired by one of the aforementioned methods.

A method applicable to the present embodiment may extract an image region of the tissue by binarizing intensity data of the image. This method is applicable in a case where a non-measuring tissue of photoacoustic measurements is detected by using illumination light having a monochromatic wavelength as described above. In this case, for the binarization, a user may be required to set a threshold of the intensity of image data. For example, the threshold may be set in advance in the measurement control unit 102 when the measurement parameter setting is performed in step 201 in FIG. 2 so that the specific region detection unit 123 may refer to the threshold. The threshold may be set in advance in the specific region detection unit 123. The threshold for the binarization may be set to a signal level of a captured image of a tissue to be visualized by performing photoacoustic measurements.

As an alternative extraction method, a user may select a region manually from a captured image of a subject. In this case, a user refers to a distinctive shape of a non-measuring tissue to extract an image of the tissue. This process may be performed by the specific region detection unit 123 in step 203, for example. This extraction method is applicable to all of the illumination methods described above.

### Method for Resetting Measurement Region

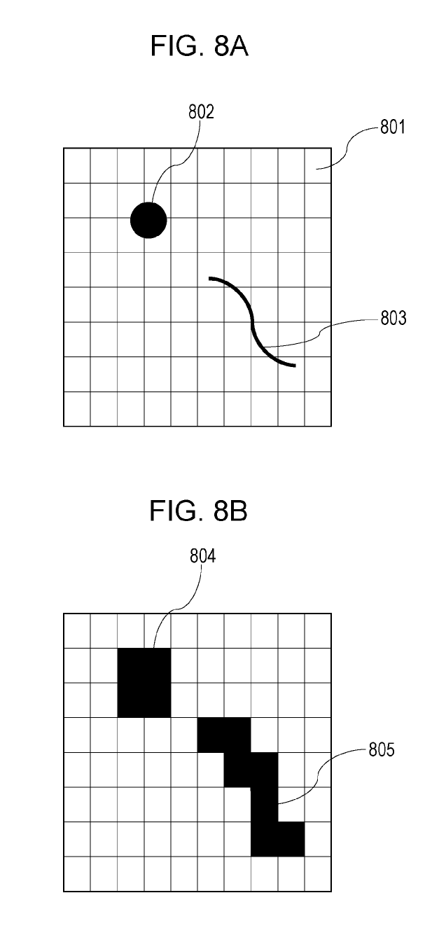

Next, with reference to FIGS. 8A and 8B, a method will be described which excludes the pixels capturing a non-measuring tissue of photoacoustic measurements, which are extracted from a captured image, from a region on which a photoacoustic measurement is performed and sets another measurement region.

FIG. 8A illustrates an example of an image acquired by superimposing image data specifying a non-measuring tissue of photoacoustic measurements, which are acquired from a captured image, on a measurement region of a two-dimensional plane on a subject 113 on which photoacoustic measurements are to be performed, viewed from a direction perpendicular to a two-dimensional image captured by the image pickup unit 122. Here, each of measurement positions on which photoacoustic measurements are to be performed corresponds to one grid 801. FIG. 8A further illustrates a mole 802 on the skin and a body hair 803 on the skin. FIG. 8B illustrates a reset measurement region. FIGS. 8A and 8B illustrate that measurement positions included in grids overlapping regions having the mole 802 and the body hair 803 are excluded from an object. In other words, a part 804 corresponding to the mole 802 is excluded from an object, and a part 805 corresponding to the body hair 803 is excluded from an object. The region corresponding to the remaining white grids is reset as a new measurement region. Following the flow illustrated in FIG. 3, it may be set such that at least one of the irradiation of pulsed light from the pulsed light source 101 and the detection of photoacoustic waves is not performed on the region excluded from the object. Alternatively, irradiation of pulsed light and detection of photoacoustic waves may be performed, or DAQ unit 110 may be prevented from performing the data saving.

The measurement region may not be divided into a grid manner but may be divided into any arbitrary shape. The method for selecting a non-measuring region of a photoacoustic wave measurement, including a tissue such as a mole or a body hair, is not limited to the method described above, but a wider region may be selected if a tissue such as a mole or a body hair is included.

### Others

The configurations and operations of the aforementioned embodiment are given for illustration purpose only and may be changed. For example, the pulsed light 104 to be irradiated to the subject 113 may be light having a wavelength absorbed by a specific one of components of the subject 113. The pulse width of the pulsed light 104 is in the order of several picoseconds to several hundred nanoseconds. If the subject is a living body, the subject may preferably absorb light having a pulse width of several to several tens nanoseconds. Though the pulsed light source 101 which generates pulsed light 104 may be a laser or may be a light emitting diode or a photographic flash lamp instead of a laser.

A laser as the pulsed light source 101 may vary such as a solid-state laser, a gas laser, a dye laser, a semiconductor laser. If pigments in which oscillation wavelengths can be changed or OPOs (Optical Parametric Oscillators) are used as light sources, differences among optical characteristic value distributions depending on wavelengths can be measured.

The wavelength of the pulsed light source 101 may be in a wavelength region of 400 nm to 1600 nm or in ranges of terahertz wave, microwave, and radio waves.

In a case where light with a plurality of wavelengths is used as the pulsed light 104, a coefficient relating to an optical characteristic within a living body is calculated and is compared with a wavelength dependency inherent to a substance (such as glucose, collagen, oxidation-reduction hemoglobin) contained in a biological tissue for each of the wavelengths. This allows imaging of concentration distributions of substances contained in the living body.

Use of the acoustic signal receiving unit allows proper visualization of a tissue that is a visualization object even in a case where a substance having a high light absorbing quantity such as a mole or a body hair is present on the skin or in an epidermal layer.

### Second Embodiment

According to a second embodiment, a method different from the first embodiment will be described which excludes measurement data of a tissue such as a mole or a body hair, which is a non-measuring of photoacoustic measurements according to the first embodiment, from data constructing an image. According to the first embodiment, a photoacoustic measurement is not performed on a non-measuring tissue. In other words, at least one of irradiation of pulsed light and detection of photoacoustic waves is not performed. According to the present embodiment on the other hand, photoacoustic measurements are performed on all of measurement regions which are initially set, and measurement data acquired from a region including a non-measuring tissue of the measurements are excluded so that the data are not to be used for constructing an image. Descriptions on the same parts as those of the first embodiment will be omitted though details will be described below, and differences from the first embodiment will be described mainly.

### Overall Configuration

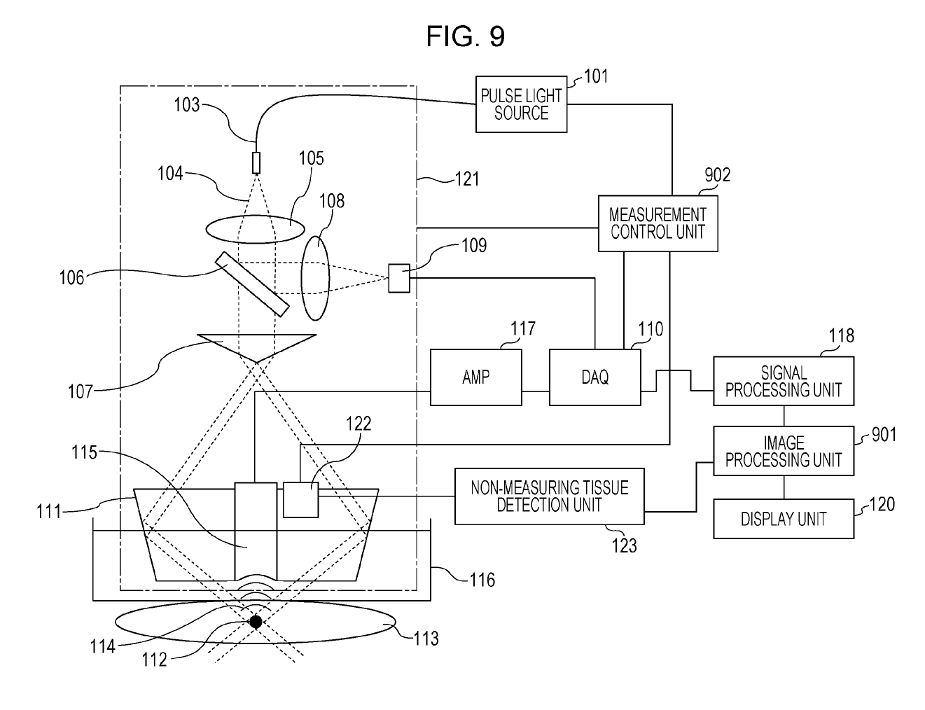

First, an overall configuration of the present embodiment will be described with reference to FIG. 9. According to the present embodiment, like the first embodiment, the specific region detection unit 123 detects a non-measuring tissue, such as a mole or a body hair, on which a photoacoustic measurement is not to be performed from an image of a subject surface captured by the image pickup unit 122. A difference from the first embodiment is that the detected information is provided to an image processing unit 901 so that data based on the non-measuring tissue may be excluded from data contributing to construction of an image. Thus, a measurement control unit 902 performs control so as to execute a photoacoustic measurement over all measurement regions set in step 201. Because the functions of the image pickup unit 122, specific region detection unit 123, and other components are the same as those of the first embodiment, the description will be omitted.

### Data Acquisition Process

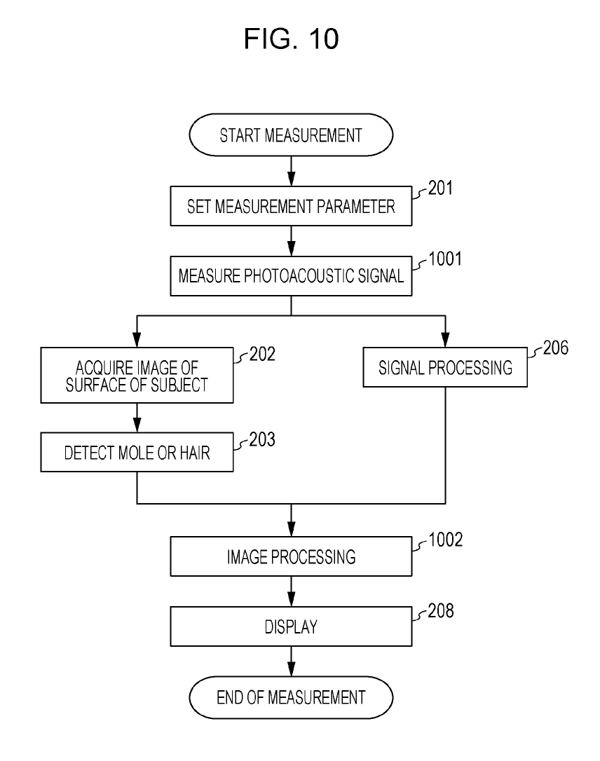

Next, a data acquisition process will be described with reference to the flowchart illustrated in FIG. 10. According to the present embodiment, a measurement parameter is set in step 201, like the first embodiment. After that, in step 1001, photoacoustic measurements are performed on all measurement positions within the measurement region set in step 201. In step 206, the signal processing unit performs signal processing on a photoacoustic signal received from the DAQ unit 110, like the first embodiment. In steps 202 and 203, images of a surface of the subject 113 captured by the image pickup unit 122 are acquired, and the specific region detection unit 123 detects a non-measuring tissue from the captured images. This method may also be performed in the same manner as that in the first embodiment. In step 1002, the image processing unit 901 converts the processed photoacoustic signal data to voxel data and excludes data based on the non-measuring tissue detected in step 203 from data constructing the images. This processing will be described in detail below. The other image processing is the same as that in the first embodiment. After the image processing is performed in step 1002, the image data are displayed by a display unit, like the first embodiment (step 208), and the measurements complete.

In this flow, data based on a non-measuring tissue is excluded from data constructing an image. The data excluded from data constructing an image may not be stored in a memory or may be stored in a memory. The region excluded from data constructing an image may be separated such that data on the region has lower reliability than measurement data on other regions and may be replaced by dummy data, for example.

### Specifying Non-Measuring Region

Because the method for detecting a non-measuring tissue such as a mole or a body hair in image data to be displayed in step 208 and the method for extracting pixel data showing such a tissue from an image captured by the image pickup unit 122 are the same as those in the first embodiment, the description will be omitted.

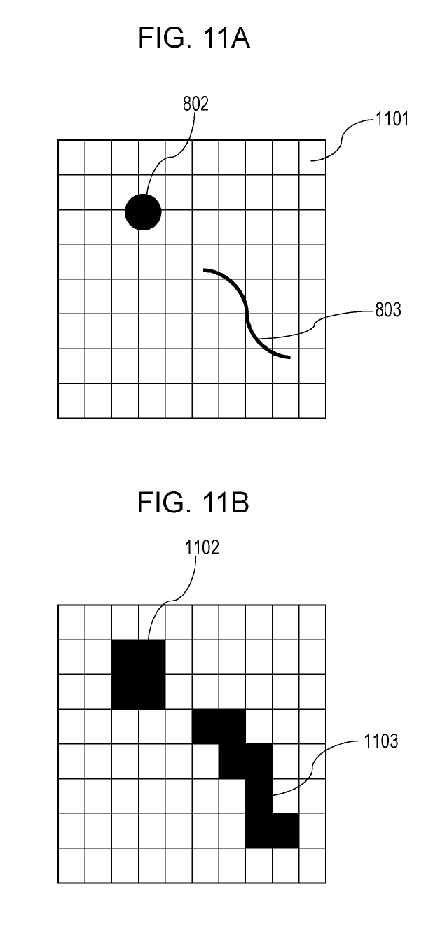

Next, a method for excluding non-measuring data from the voxel data generated from a measured photoacoustic signal in step 1002 will be described with reference to FIGS. 11A and 11B.

First, like the first embodiment, the positional alignment of an image captured by the image pickup unit 122 and a region set as a photoacoustic measurement region is adjusted. FIG. 11A illustrates an image acquired by superimposing image data specifying a non-measuring tissue of photoacoustic measurements, which are acquired from a captured image, on a measurement region of a two-dimensional plane on the subject 113 on which photoacoustic measurements are to be performed, viewed from a direction perpendicular to a two-dimensional image captured by the image pickup unit 122. Here, each of positions on which photoacoustic measurements are to be performed within the measurement region set in step 201 corresponds to one grid 1101. As described above, in step 1002 of the present embodiment, photoacoustic measurement data are acquired from all measurement positions. Then, voxel data generated from measurement data included in a grid including a non-measuring tissue is excluded so that the data are not to be used for constructing an image. In other words, as illustrated in FIG. 11B, the voxel data generated from measurement data on regions included in grids 1102 and 1103 including the mole 802 and the body hair 803 of the non-measuring tissue are excluded. The processing in step 1002 has been described up to this point.

The method for dividing a measurement region is not limited to the aforementioned example, but a measurement region may be divided into arbitrary shapes. The method for selecting a region excluded from the voxel data, including a tissue such as a mole or a body hair, is not limited to the method described above, but a wider region may be selected if a tissue such as a mole or a body hair is included.

The present embodiment may also apply the configuration according to the first embodiment.

The acoustic signal receiving unit according to the present embodiment allows proper visualization of a tissue that is a visualization object even in a case where a substance having a high light absorbing quantity such as a mole or a body hair is present on the skin or in an epidermal layer.

### Third Embodiment

According to a third embodiment, photoacoustic tomography (PAT) is applied which acquires a sound pressure of a photoacoustic wave generated from a subject at a plurality of positions on a two-dimensional plane and performs an image reconstruction calculation based on it to generate three-dimensional information relating to optical characteristic values of inside of the subject. This is different from the method applied in a photoacoustic microscope which performs photoacoustic measurements at a plurality of positions within a limited measurement region from which photoacoustic waves may be measured in the measurements by focusing light or sound waves to generate three-dimensional information as in the first embodiment and the second embodiment.

### Overall Configuration

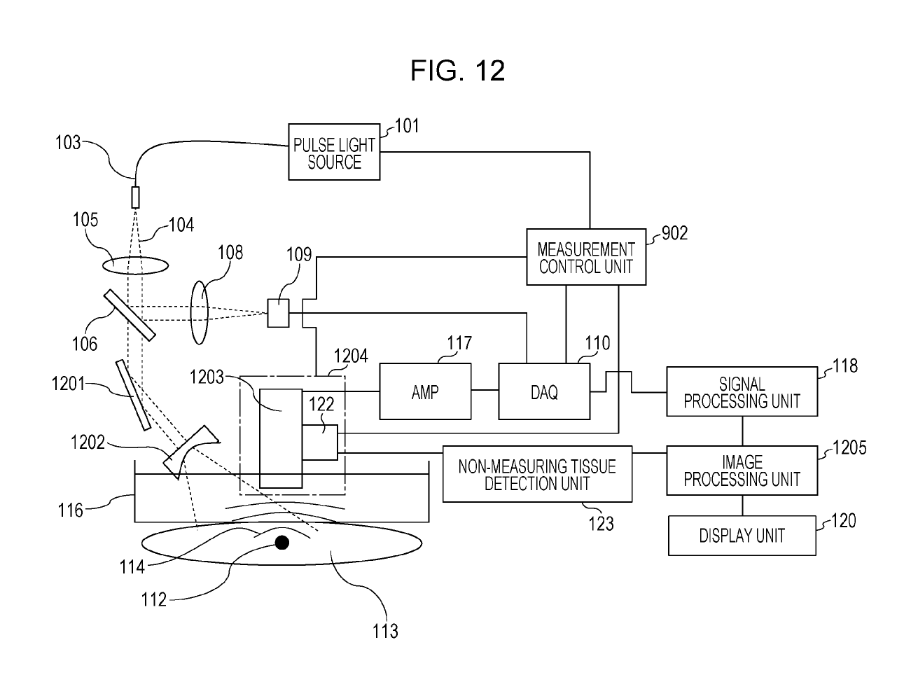

A configuration of the present embodiment will be described with reference to FIG. 12. Like numbers refer to like parts in the descriptions of the first and third embodiments, and repetitive description will be omitted. According to the present embodiment, pulsed light 104 for exciting photoacoustic waves is generated from the pulsed light source 101, like the first embodiment. After the pulsed light 104 passes through the beam splitter 106, the pulsed light 104 is irradiated to the subject 113 through a mirror 1201 and a concave lens 1202. Unlike the first and second embodiments, the pulsed light 104 is spread by the concave lens 1202 and is irradiated to a subject according to the present embodiment. The irradiated pulsed light 104 excites photoacoustic waves 114 from the light absorber 112 within the subject, and a transducer 1203 detects the sound pressure of the photoacoustic waves 114. The transducer 1203 may be an ultrasonic transducer sensitive to an ultrasonic frequency band. According to the present embodiment, in order to move the transducer 1203 and image pickup unit 122 that are enclosed within a box 1204 two-dimensionally by a movable stage (not illustrated), an optical system for guiding the pulsed light 104 for exciting photoacoustic waves to the subject 113 is fixed. The scanning by using the movable stage allows acquisition of the photoacoustic waves 114 at a plurality of positions on a two-dimensional plane. The transducer 1203 of the present embodiment does not include an acoustic lens. Therefore, photoacoustic waves generated in a wider region may be detected instead of selective detection of photoacoustic waves generated in a region on which light or sound waves are focused as in the first or second embodiment. An image processing unit 1205 according to the present embodiment calculates an initial pressure distribution of photoacoustic waves within the subject 113 from a photoacoustic signal acquired on the two-dimensional plane by performing image reconstruction. The image processing unit 1205 further excludes data on a tissue not to be displayed, which are detected by the specific region detection unit 123, from data used for the image reconstruction based on positional information of the tissue within a photoacoustic measurement region, like the second embodiment. Then, the photoacoustic image data acquired by performing image processing are displayed by a display unit.

### Data Acquisition Process

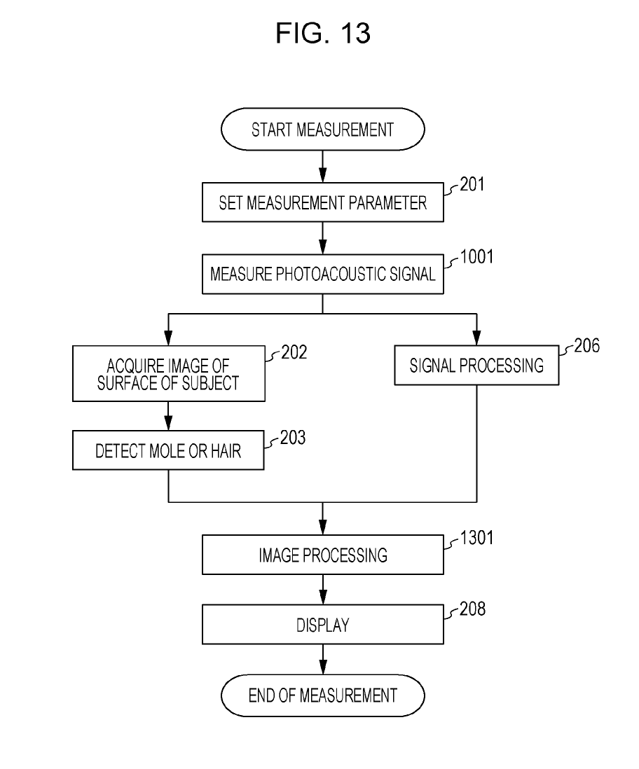

Next, a data acquisition process will be described with reference to FIG. 13. The third embodiment is different from the first and second embodiments in that, in step 1301, the image reconstruction calculation is performed within the image processing unit 1205 to generate voxel data of an initial pressure distribution of photoacoustic waves within the subject. After that, within step 1301, measurement data based on a tissue in the specific region detected in step 203 are excluded from voxel data.

According to the present embodiment, measurement data not to be measured are excluded from the voxel data. The data excluded from data constructing an image may not be stored in a memory or may be stored in a memory. The excluded region may be separated such that data on the region has lower reliability than measurement data on other regions and may be replaced by dummy data, for example.

### Specifying Non-Measuring Region

Because the method for detecting a non-measuring tissue, such as a mole or a body hair, in image data to be displayed finally and the method for extracting pixel data showing such a tissue from an image captured by the image pickup unit 122 are the same as those in the first embodiment, the description will be omitted.

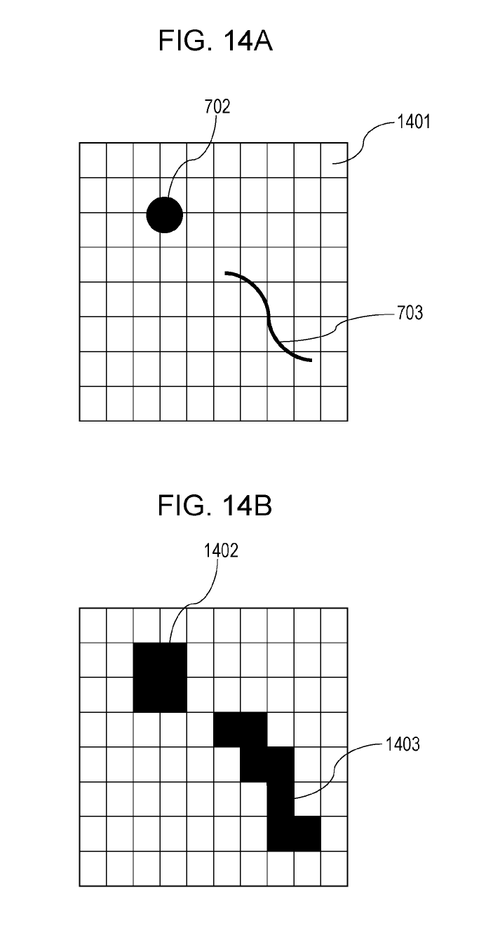

Next, a method for excluding data not to be measured from the voxel data generated from a measured photoacoustic signal in step 1301 will be described with reference to FIGS. 14A and 14B.

First, the positional alignment of an image captured by the image pickup unit 122 and voxel data generated by the image reconstruction calculation in step 1301 is adjusted. FIG. 14A illustrates an image acquired by superimposing a captured image on positions of voxels. FIG. 14A is a view from a direction perpendicular to a two-dimensional image captured by the image pickup unit 122. The illustrated grids correspond to voxels projected in the direction. A grid 1401 represents a minimum voxel having undergone the reconstruction calculation. On the superimposed image, voxel data having a grid including a non-measuring tissue as its upper surface are excluded. In other words, as illustrated in FIG. 11B, voxel data having grids 1402 and 1403 including the mole 802 and the body hair 803 which are non-measuring tissues as its upper surface and being present in the vertical direction of FIG. 11B are excluded. The processing in step 1301 has been described up to this point.

Having described above that measurement data on a non-measuring tissue are excluded from the voxel data, the excluded data may not be stored in a memory or may be stored in a memory. The region on which the excluded data are based may be separated such that data on the region has lower reliability than measurement data on other regions, and dummy data, for example, may be included. The method for dividing a measurement region into voxels used for the reconstruction calculation is not limited to the aforementioned method, but a measurement region may be divided into arbitrary shapes. The method for selecting a region excluded from the voxel data, including a tissue such as a mole or a body hair, is not limited to the method described above, but a wider region may be selected if a tissue such as a mole or a body hair is included.

The present embodiment may also apply the configuration according to the first embodiment. According to the present embodiment, the transducer 1203 is moved on a two-dimensional plane by a movable stage (not illustrated) to perform a photoacoustic measurement. However, a plurality of transducers may be arranged as a two-dimensional array to measure photoacoustic signals on the two-dimensional plane without performing the scanning by the movable stage. Use of this configuration may increase the speed of the measurements.

Use of the acoustic signal receiving unit according to the present embodiment allows proper visualization of a tissue that is a visualization object even in a case where a substance having a high light absorbing quantity such as a mole or a body hair is present on the skin or in an epidermal layer.

The aforementioned embodiments are given for illustration purpose only and may be combined with each other without departing from the spirit and scope of the present invention.

The aforementioned photoacoustic measurement apparatus may be utilized as a medical diagnostic imaging apparatus in a case where a subject is a biological substance. More specifically, for diagnoses and follow-up studies of chemical treatments performed on tumors and vascular diseases, an optical characteristic value distribution within a living body and a concentration distribution of a substance contained in a biological tissue from the information may be imaged.

The present invention is applicable to a non-destructive test to be performed on a non-biological substance.

The present invention allows proper visualization of the skin or an epidermal layer even in a case where a substance having a high light absorbing quantity such as a mole or a body hair is present on the skin or in the epidermal layer.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-178561, filed Sep. 2, 2014, which is hereby incorporated by reference herein in its entirety.

What is claimed is:

1. A photoacoustic wave measurement apparatus comprising:
an image pickup unit configured to capture a surface image of an object;
a light source configured to generate light;
a photoacoustic wave detection unit configured to detect photoacoustic waves propagated from an object irradiated with the light;
a specific region detection unit configured to detect a specific region within the captured surface image of the object; and
a control unit,
wherein the control unit sets a measurement region of the object to perform irradiation of the light and controls detection of the photoacoustic waves based on positional information of the specific region detected by the specific region detection unit.

2. The photoacoustic wave measurement apparatus according to claim 1, further comprising an image processing unit configured to generate image data based on the photoacoustic waves detected by the photoacoustic wave detection unit.

3. The photoacoustic wave measurement apparatus according to claim 2, further comprising a display unit configured to display an image based on the image data generated by the image processing unit.

4. The photoacoustic wave measurement apparatus according to claim 1, wherein the control unit sets the measurement region such that the irradiation of the light and the detection of the photoacoustic waves are performed in a region of the object corresponding to a region excluding the specific region of the captured image.

5. The photoacoustic wave measurement apparatus according to claim 1, wherein the control unit sets the measurement region such that at least one of the irradiation of the light and the detection of the photoacoustic waves is not performed in a region on the object corresponding to a region from which the specific region of the captured image is detected.

6. The photoacoustic wave measurement apparatus according to claim 1, wherein the light source generates the light in both of a case where the captured image is acquired by the image pickup unit and a case where the photoacoustic waves are measured by the photoacoustic wave detection unit.

7. The photoacoustic wave measurement apparatus according to claim 6, wherein the light source generates light having an identical wavelength characteristic in both of the case where the captured image is acquired by the image pickup unit and the case where the photoacoustic wave detection unit measures the photoacoustic waves.

8. The photoacoustic wave measurement apparatus according to claim 6, wherein the light source generates light having wavelength characteristics different from each other in the case where the captured image is acquired by the image pickup unit and the case where the photoacoustic wave detection unit measures the photoacoustic waves.

9. The photoacoustic wave measurement apparatus according to claim 1, wherein
the light source generates a plurality of kinds of light having mutually different wavelength characteristics by switching;
the image pickup unit obtains the captured image in a state of irradiating the plurality of kinds of light to the object; and
the specific region detection unit detects the specific region by calculating a correlation coefficient based on the captured image acquired in a state of irradiating the plurality of kinds of light to the object.

10. The photoacoustic wave measurement apparatus according to claim 1, further comprising a stage having the photoacoustic wave detection unit thereon and being capable of relative movement about the object.

11. The photoacoustic wave measurement apparatus according to claim 1, further comprising an optical unit configured to guide the light to the object.

12. A photoacoustic wave measurement apparatus comprising:
an image pickup unit;
a light source configured to generate light;
a photoacoustic wave detection unit configured to detect photoacoustic waves propagated from an object irradiated with the light;
a specific region detection unit configured to detect a specific region within a captured image obtained by the image pickup unit; and
an image processing unit,
wherein the image processing unit generates image data from the photoacoustic waves detected by the photoacoustic wave detection unit based on positional information of the specific region detected by the specific region detection unit.

13. The photoacoustic wave measurement apparatus according to claim 12, wherein, with respect to a position within the object corresponding to the specific region,
the image processing unit excludes, from the image date, data generated based on the photoacoustic waves detected at the position within the object.

14. The photoacoustic wave measurement apparatus according to claim 13, wherein the data excluded from the image data is replaced by dummy data.

15. The photoacoustic wave measurement apparatus according to claim 1, wherein the specific region detection unit determines as the specific region, one of a pixel having a higher signal level than a threshold and a pixel having a lower signal level than the threshold among a plurality of pixels constructing the captured image.

16. The photoacoustic wave measurement apparatus according to claim 15, wherein the specific region is the pixel having the signal level lower than the threshold among the plurality of pixels constructing the captured image.

17. A photoacoustic wave measurement method comprising:
obtaining a captured image of an object;
detecting a specific region within the captured image;
setting a measurement region of the object based on a position of the specific region within the captured image; and
irradiating light to the measurement region and detecting photoacoustic waves propagated from the measurement region of the object in response to the irradiation of the light.

18. The photoacoustic wave measurement method according to claim 17, wherein the irradiation of the light and the detection of the photoacoustic waves are performed in a region of the object corresponding to a region excluding the specific region of the captured image.

19. The photoacoustic wave measurement method according to claim 17, wherein at least one of the irradiation of the light and the detection of the photoacoustic waves is not performed in a region of the object corresponding to a region from which the specific region of the captured image is detected.

20. The photoacoustic wave measurement method according to claim 17, wherein one of a pixel having a higher signal level than a threshold and a pixel having a lower signal level than the threshold among a plurality of pixels constructing the captured image is selected as the specific region.

21. The photoacoustic wave measurement method according to claim 20, wherein the pixel having the signal level lower than the threshold among the plurality of pixels constructing the captured image is selected as the specific region.

22. The photoacoustic wave measurement method according to claim 17, further comprising:
irradiating a plurality of kinds of light having mutually different wavelength characteristics to the object and acquiring the captured image in a state of irradiating the plurality of kinds of light to the object; and
calculating a correlation coefficient based on the captured image acquired in the state of irradiating the plurality of kinds of light to the object,
wherein the detecting comprises detecting the specific region based on the correlation coefficient.

23. A photoacoustic wave measurement method comprising:
obtaining a captured image of an object;
detecting a specific region within the captured image;
irradiating light to the object and detecting photoacoustic waves propagated from the object in response to the irradiation of the light; and
generating image data from the detected photoacoustic waves based on a position of the specific region within the captured image.
